(19) United States
(12) Patent Application Publication
(10) Pub. No.: US 2022/0126070 A1
(43) Pub. Date: Apr. 28, 2022

(54) ARTERIAL BALLOON WITH VARIABLE PRESSURES

(71) Applicant: Mohamed Abdalla Mahmoud Eltahlawi, Zagazig (EG)

(72) Inventor: Mohamed Abdalla Mahmoud Eltahlawi, Zagazig (EG)

(21) Appl. No.: 17/431,604

(22) PCT Filed: Sep. 1, 2019
(86) PCT No.: PCT/EG2019/000018

§ 371 Date: Aug. 17, 2021

(30) Foreign Application Priority Data
Feb. 20, 2019 (EG) 2019020282

(51) Int. Cl. A61M 25/10; A61F 2/958; A61M 29/02

(52) U.S. Cl. CPC A61M 25/1011; A61M 25/104; A61F 2/958; A61F 2002/9583; A61M 29/02; A61M 2205/3344; A61M 2025/1013; A61M 25/10184

(57) ABSTRACT

Provided is a balloon with multiple small segments (balloons) fused to each other along the shaft of the balloon. Each segment has a different pressure profile. Each segment has a separate outlet (slot) used for its inflation with the required pressure according to the tightness and calcification of the lesion of the arterial wall. Each segment can be inflated with different pressures and the apposition of the overlying stent can be confirmed without the need for another non compliant balloon which carry many complications and add extra cost.

### CROSS-REFERENCE TO RELATED APPLICATIONS

This application claims priority to PCT Application No. PCT/EG2019/000018, having a filing date of Sep. 1, 2019, based on EG Application No. 2019020282, having a filing date of Feb. 20, 2019, the entire contents both of which are hereby incorporated by reference.

### FIELD OF TECHNOLOGY

The following is in the field of medical engineering. It is concerned with medical equipment used in cardiovascular catheterization.

### BACKGROUND

Narrow and stenosed arteries are dilated by the interventional catheterization techniques and under the catheter device (cath lab). This is done by using balloons or by stenting the artery using metallic stents to keep the artery open. The doctor inserts a catheter that carries a small balloon into the artery through the arteries of the thigh or arm and directs it to the place of constriction in the coronary artery, for example. The stents also are installed (mounted) on balloons. The diameter of the stent increases gradually with increased the pressure of its balloon inflation up to a certain extent (the maximum burst pressure) after which the balloon bursts.

The balloon is made of special material that is resistant to pressure, cracking or explosion and sometimes withstands 20 times the value of atmospheric pressure. These materials are all compatible with the body such as polyethylene or nylon.

The balloons used in the treatment and expansion of narrow blood vessels could be balloons with low pressure, medium pressure, or high pressure. That refers to the amount of pressure needed to inflate such balloon before it bursts. Stents used in the treatment of these lesions (narrowing) are also installed on balloons with different pressures.

The empty balloon catheter is placed, then inflated to perform the necessary procedure, and deflated again before removal. When the balloon is inflated it expands the stents. When the balloon is emptied later, the stent remains against the arterial wall and the balloon catheter could be then removed. This depends on the pressure needed to inflate the balloon. Each of these balloons is used according to the narrowness and calcification of the site of injury in the artery with the necessary pressure increases the greater the narrowing or calcification on the affected part of the artery.

Each balloon has a minimum pressure that allows it to be extended to the radius assigned to it and has a maximum pressure that will explode the balloon if exceeded.

The balloons could be compliant (its diameter increases continuously with increased pressure of inflation but with maintaining low internal pressure) and non compliant (i.e.,, increasing the internal pressure of the balloon with increased inflation pressure but without considerable increase in diameter, allowing the dilatation of tightly stenosed or heavily calcified areas of the arteries).

The problem or shortcomings in the previous art:

In many cases, the diameter of the artery differs in the upper part of the artery from the lower part. In other cases, the diseased segment of the artery has different severity and calcification along its whole length. Therefore, the choice of the diameter of the needed balloon and the amount of pressure that the balloon inflates is confusing and problematic.

If the balloon is inflated with low pressure suitable for the small part of the artery, the widely large segment of the artery will not benefit from balloon inflation. If the balloon is inflated at high pressure, the diameter of the balloon and stent will increase more than the diameter of the small part of the artery, which may cause rupture of the artery.

Therefore, doctors use a balloon (or a balloon-mounted stent) with low pressure that partially opens the narrowness first to widen the narrow part of the artery and then another higher pressure balloon is used to expand the part of the artery with the wider diameter.

The type of balloon in this case is called (non-compliant) which has a length similar to the remaining narrowing length to complete the expansion of the artery and confirm the positioning of the stent completely against the arterial wall.

More than one balloon may be used with different pressures to expand the different parts of the artery with different diameters and different calcification ratio. This increases the cost and carries a risk each time to rupture the artery.

In some cases, an internal fracture of the implanted stent may occur depending on the quality of the stent and the anatomical area in which this stent is located example, in the “below knee” region which moves periodically and in different directions.

There are some previous arts (balloon catheters) consisting of multiple balloons:

A. Regarding “Multiple balloon application catheter (EP0266957A2):

It is a catheter with 2 balloons at its distal end, one inside the other with separate lumen for each balloon. An inner balloon mounted on the distal end of the catheter body, and an outer balloon mounted on the distal end of the catheter body and inclosing the inner balloon. While a first inflation lumen has a distal end in communication with the interior of the outer balloon, and a second inflation lumen has a distal end in communication with the interior of the inner balloon.

The inflation of both balloons could not occur simultaneously at the same time, while each balloon is inflated separately and independently on the other.

There are different legs for inflation of each balloon using the inflation devices.

B. Regarding “Angle inflatable composite balloon & method” (US20130238038A1):

This invention is designed for use in bone surgery. It is made of metal, ceramic or synthetic biological material.
The device is comprising a tube having a longitudinal axis with a distal end portion and a polarity of inflatable balloons coupled longitudinally in series along the distal end portion of the tube. The balloons could be inflated and controlled by single syringe or independently inflated by multiple devices. The inflation of each balloon is done through different path so that each balloon should be inflated separately at a time. The inflation device (or devices) fluidly communicate with the balloons through lumen. There may be one or more paths for fluid communications with balloons through the lumen. The controlled inflation volumes are controlled using syringe for each balloon.
The plurality of each balloon being configured to have individually controlled infusion volumes, the plurality of inflatable balloon including different dimensions such that upon inflation of the balloons a composite profile shape is achieved.

C. Tandem independently inflatable/deflatable multiple diameter balloon angoplasty catheter systems and method of use (U.S. Pat. No. 4,763,654):

It is a multi-lumen catheter bearing a plurality of individually inflatable and deflatable balloons of predetermined, different sizes. The balloons are mounted in tandem on the catheter shaft so that a smaller balloon is bonded to the distal end of the catheter just distally of a larger, separately inflatable balloon. A catheter for performing balloon angioplasty, comprising:

an elongate, flexible catheter shaft having at least two lumens therein; at least two relatively inelastic angioplasty balloons on said shaft, wherein said balloons are connected to separate lumens for independent inflation and deflation thereof, and each of said balloons has a proximal end, a distal end. The catheter, further comprising an annular partition of balloon material in a tube having an inner edge and an outer edge, wherein said outer edge is bonded to said tube and said inner edge is bonded to said catheter shaft to separate the interior of said first balloon from the interior of said second balloon.

D-Novel muti-segment balloon dilatation catheter (CN102068749A). It is a multisection type of expandable balloon catheter, comprises seal wire, inner catheter, outer catheter, sacculus with different sizes and development ring. The multistage saccules are arranged in ascending, and perhaps descending size manner.

E-SEGMENTED BALLOON CATHETER FOR STENTING BIFURCATION LESIONS (U.S. Pat. No. 6,761,734B2)

A segmented balloon catheter for use in treating a lesion of a vessel occurring near a bifurcation that is defined by the intersection of a main vessel with a side branch vessel. The segmented balloon catheter comprises a shaft which includes a proximal end, a distal end and a longitudinal passageway that extends therethrough from the proximal end to the distal end.

### SUMMARY

An aspect relates to a balloon made up of many small balloons that merge with each other along a balloon column. The column contains multiple slots (ports) that can be detected separately during blowing so that the blowing pressure is directed to only one slot at a time.

(This feature is not present in any of the abovementioned previous balloons).

Each part has different pressure properties so that different tolerable pressures are likely.

Each section has a separate outlet that is used to inflate it with the desired pressure according to the narrowing and calcification of the corresponding arterial wall.

Each part can be inflated individually with different pressures according to need and calcification ratio and the placement of the local stent against the wall of the artery and adjacent to it can be confirmed completely without the need for other balloons that are non-compliant with many complications and add additional cost.

### BRIEF DESCRIPTION

Some of the embodiments will be described in detail, with references to the following Figures, wherein like designations denote like members, wherein:

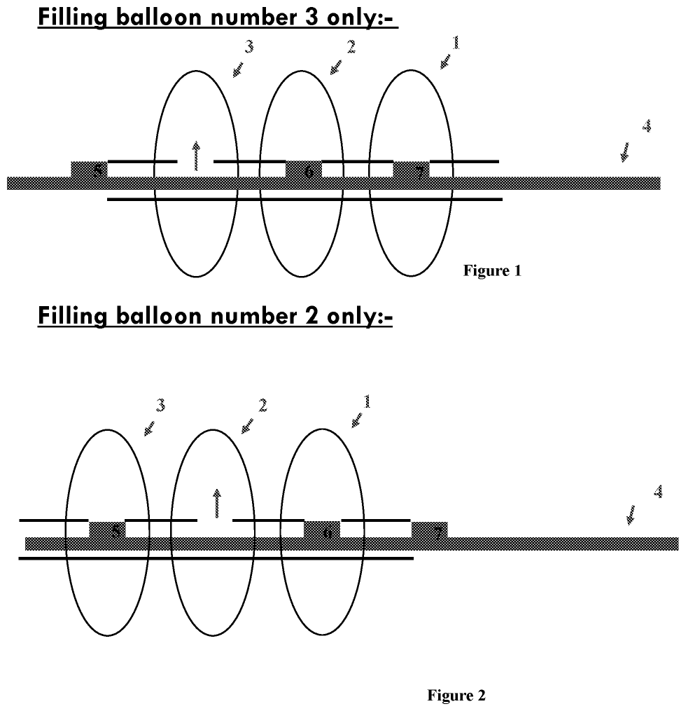

FIG. 1 depicts a balloon according to embodiments of the invention in which only a third balloon is filled;

FIG. 2 depicts the balloon according to embodiments of the invention in which only a second balloon is filled;

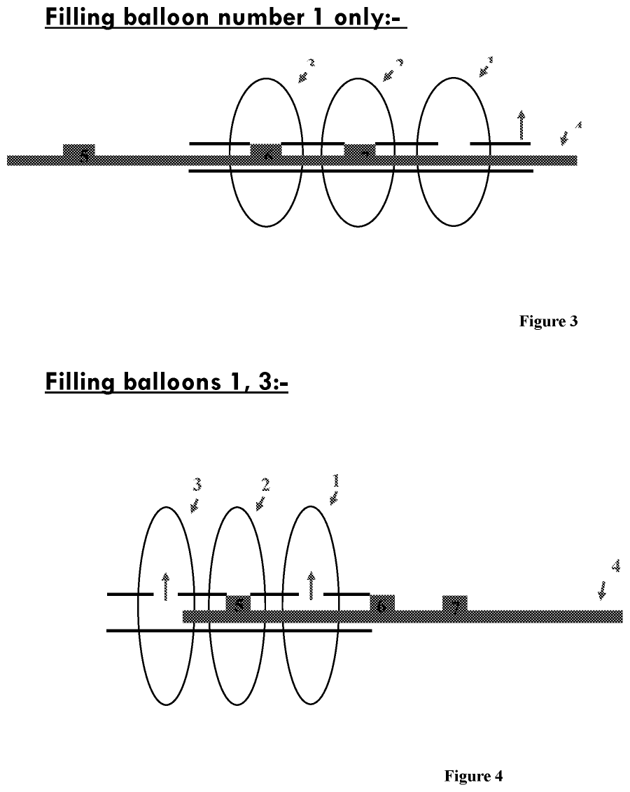

FIG. 3 depicts the balloon according to embodiments of the invention in which only a first balloon is filled;

FIG. 4 depicts the balloon according to embodiments of the invention in which the first balloon and the third balloon are filled;

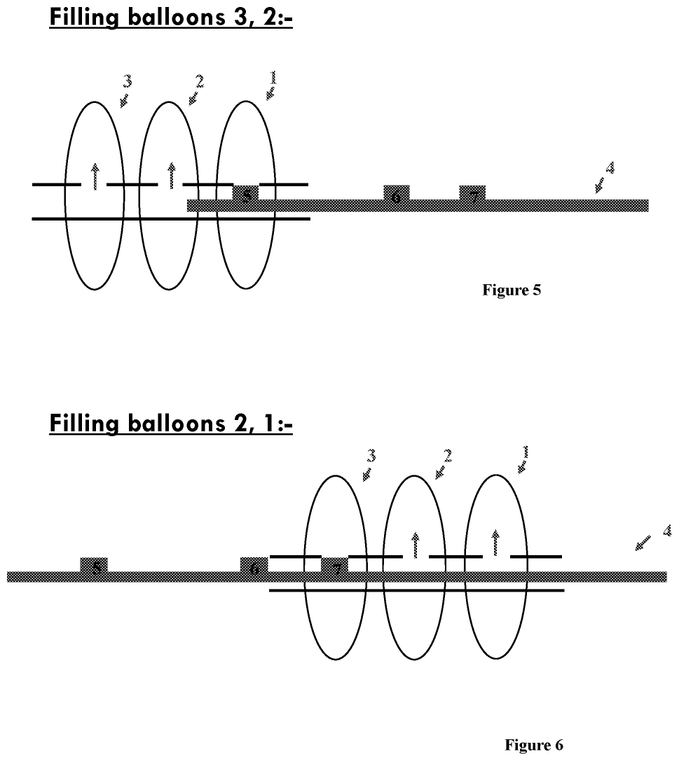

FIG. 5 depicts the balloon according to embodiments of the invention in which the second balloon and the third balloon are filled;

FIG. 6 depicts the balloon according to embodiments of the invention in which the first balloon and the second balloon are filled; and

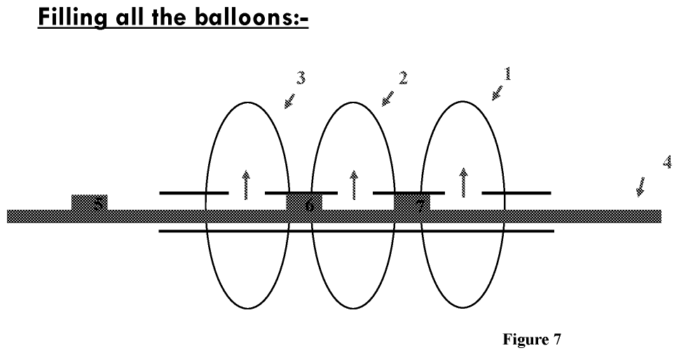

FIG. 7 depicts the balloon according to embodiments of the invention in which all of the first balloon, second balloon, and the third balloon are filled.

### DETAILED DESCRIPTION

Referring to the Figures, the following elements are shown:
1—The first balloon;
2. Second balloon;
3. The third balloon;
4 The sliding column (arm) of inflation which is connected to the endflator;
5. First (distal) protrusion;
6. Second (middle) protrusion (at a distance X from the first one (no 5));
7. Third (distal) protrusion (at a distance from the middle projection (no 6) equal to 1/2×);
8. Markers and locks to on the external surface of the sliding column (arm) to lock the movement of the sliding column when the desired distance is reached; and
9. Endflator
   The balloon usually consists of a column with a cavity that allows for a very thin guide wire (for example, 0.014 inches).
   In the arm there is another cavity used to inflate the balloon, which is usually used with saline solution (not air).
   At the end of the balloon arm, there is an inflator.

The balloon consists of a group of 3-5 small balloons adjoining each other, but their cavities do not overlap. Each balloon has a length range (5-10 mm) and diameter range (2-4.5 mm) in the embodiment used in coronary angioplasty.
In the embodiment of the current invention used in peripheral angioplasty, each balloon should have a length range (5-20 mm) and diameter range (5-15 mm).
Each small balloon has the ability to expand according to the amount of pressure used for its inflation, so that it expands alone without the other adjacent parts (balloons).
The arm at the end of the balloon has several adjacent openings at regular intervals, each of which corresponds to the balloon facing it.

(These openings are aligned along single inflation path that makes embodiments of this invention different from, for example, “Angle inflatable composite balloon & method” (US20130238038A1) discussed above, in which the inflation of each balloon is done through different path so that each balloon should be inflated separately at a time.
The front of the arm (column) cavity where the balloon is inflated on a thin metal wire (control wire) has at its end 3 protrusions on regular interval distance except for the third which is on double the interval distance.

(This feature is not present in any of the previous arts).

### Method of Operation

1—When inflating the balloon, 2 protrusions will be closing the opposite balloons at a time, so that only one balloon will be inflated. When inflating 2 balloons, the column is retracted so that only one protrusion closes the opposing balloon. On inflating the 3 parts, the column is retracted in a way that all the protrusions are away from the opposing openings. Therefore one can inflate whatever balloons are needed at a time with a single inflator.

(This feature is not present in any previous art. For example, The Angle inflatable composite balloon & method” (US20130238038A1) discussed above requires each balloon to be inflated separately at a time. Therefore the operator needs multiple inflators for inflation all balloons together at a time).

2. The inflating pressure of each balloon is controlled by the endflator (as is the case in all currently used balloons), but each small balloon is inflated according to different needs and pressure.

3. When the balloon is blown up (or the stent is installed), the balloons are completely inflated by pulling the control wire as described above so that all the openings are exposed and it is easy to inflate the balloons together.

4. Inflation of both the proximal and distal balloons can have an anchoring effect preventing sliding of the balloon over the calcified lesion while inflation the middle balloon(s).

### Method of Exploitation of the Possible Embodiments

1—Balloon of dilatation of coronary arteries (PTCA balloon): in this case each balloon has a length range (5-10 mm) and diameter range (2-4.5 mm) 2—Balloon dilatation of the peripheral arteries: in this case in peripheral angioplasty, each balloon should have a length range (5-20 mm) and diameter range (5-15 mm).

3—Stents could be mounted on the balloon to be implanted in the coronary or peripheral arteries.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A catheter for performing vascular angioplasty comprising:
an inflating balloon catheter having multiple balloons, wherein the multiple balloons include at least three balloon, wherein each balloon of the multiple balloons adjoin another balloon without overlapping cavities, and wherein each said balloon of the multiple balloons has the ability to expand individually and separately such that each balloon of the multiple balloons can be inflated alone;
wherein the inflating balloon catheter is configured for mounting of a stent and at least one balloon of the multiple balloons is configured for delivering the stent,
wherein the inflating balloon catheter has a catheter shaft with a cavity for a guide wire;
wherein in the catheter shaft there is a second cavity used to inflate at least one balloon of the multiple balloons;
wherein at an end of the catheter shaft, there is a connection for an inflator;
wherein separate
openings within the catheter shaft are provided to inflate each balloon of the multiple balloons separately;
wherein a sliding column is provided within the catheter shaft for inflation of at least one balloon of the multiple balloons; and
wherein the sliding column carries multiple protrusions configured to close openings of the multiple balloons during inflation.

2. The catheter of claim 1, wherein each balloon of the multiple balloons is formed of a single, continuous tube of polymer material and wherein each balloon of the multiple balloons is configured to accept a mount of a stent.

3. The catheter of claim 1, wherein the separate openings are present at the end of the catheter shaft at regular intervals and each respective opening of the separate openings connects to a balloon of the multiple balloons facing the respective opening.

4. The catheter of claim 1, wherein the sliding column controls the openings of the multiple balloons.

5. The catheter of claim 1, wherein the sliding column controls the openings of each balloon of the multiple balloons independently.

6. The catheter of claim 1, wherein the connection for the inflator is used for all balloons of the multiple balloons.

7. The catheter of claim 1, further comprising labels on the catheter shaft configured for guiding the movement of the sliding column for inflation of respective balloons of the multiple balloons.

8. A method for performing dilation of a target vessel having a target lesion with different diameters or different calcifications along its length, comprising:
providing the catheter of claim 1;
placing the catheter such that at least one balloon of the multiple balloons is
inside the target vessel opposite the target lesion;
controlling the sliding column; and
controlling an inflating pressure by the inflator.
